(19) United States
(12) Patent Application Publication
(10) Pub. No.: US 2007/0264335 A1
(43) Pub. Date: Nov. 15, 2007

(54) Modified release tablets comprising tramadol

(76) Inventor: Bernard Charles Sherman, Toronto (CA)

Correspondence Address: NIXON & VANDERHYE, PC, 901 NORTH GLEBE ROAD, 11TH FLOOR, ARLINGTON, VA, 22203, US

(21) Appl. No.: 11/430,156

(22) Filed: May 9, 2006

(51) Int. Cl. A61K 31/137; A61K 9/24

(52) U.S. Cl. 424/472; 514/650

(57) ABSTRACT

Tablets for once-daily administration comprising a core comprising tramadol or a salt thereof, and a coating applied to said core comprising at least one water-insoluble polymer and at least one enteric polymer.

### BACKGROUND OF THE INVENTION

Tramadol, first described in U.S. Pat. No. 3,652,589, is an orally active opiod analgesic. Immediate release tablets containing tramadol, as its hydrochloride salt, have been available for many years.

More recently, extended release tablets have been available.

For example, tablets for twice daily administration are available in Europe under the tradename Zydol SR™; and tablets for once daily administration are available in Europe under the tradenames Zydol XL™, Xamdol XL™ and Tramador™, and in the United States under the tradename Ultram ER™.

Technologies for the manufacture of tablets containing tramadol or a salt thereof suitable for once daily administration are disclosed in U.S. Pat. No. 5,591,452, Canadian patent application no. 2,489,855, and U.S. patent application Ser. No. 10/434,266.

Each of the technologies disclosed in these publications has deficiencies.

Tablets disclosed in U.S. Pat. No. 5,591,452 comprise tramadol or a salt thereof in a controlled release matrix. The amounts of excipients (inactive) ingredients in these tablets are relatively large, with the result that the tablets are relatively large.

The tablets disclosed in Canadian patent application 2,489,855 are compression-coated tablets, which also contain relatively large amounts of excipients, with the result that the tablets again are relatively large; and moreover, the compression-coating process of manufacture is also relatively complex.

™ Trademark.

U.S. patent application Ser. No. 10/434,266 discloses tablets comprising tramadol hydrochloride that are suitable for once daily administration and are relatively small. The small size is a result of the tablets being made as core tablets that are immediate release (and thus do not require large amounts of excipients to retard release), and the control of release is achieved by applying to the cores a relatively thin film coating that retards release. This film coating comprises at least one polymer that is water-insoluble but water-permeable, at least one plasticizer, and at least one water-soluble polymer. These tablets achieve a specified dissolution profile as measured in 0.1 N HCl.

This technology is also constraining, because the requirement to use, in the film coat, both a water-insoluble polymer and a water-soluble polymer, and the requirement to achieve a specific dissolution profile as measured in 0.1 N HCl limits the polymers that can be used. This makes it difficult, for example, to find suitable coating systems that can be sprayed onto the cores as aqueous latex dispersions. It is presumably for this reason that, in all of the examples of this publication, one or more alcohols are used as solvents in the coating process.

In light of this prior art, the objective of the present invention is to enable tablets containing tramadol or a salt thereof that are suitable for once daily administration, that are relatively small in size, and that are made as a rapid-release core to which a film-coating is applied to slow release, but where the dissolution is not required to meet a specified profile in 0.1 N HCl and wherein there is wider range of polymers that may comprise the film coating.

### DESCRIPTION OF THE INVENTION

The tablets of the present invention are tablets for once-daily administration comprising (i) a core comprising tramadol or a salt thereof, preferably tramadol hydrochloride; and (ii) a coating applied to said core comprising at least one water-insoluble polymer and at least one enteric polymer.

In addition to the tramadol or salt thereof, the core will also comprise at least one excipient. The total amount of excipients by weight in the core will preferably be less than 50% of the amount of tramadol or salt thereof, more preferably less than 30% and even more preferably less than 20%.

“Water-insoluble” will be understood to mean insoluble in water regardless of pH.

An “enteric polymer” will be understood to mean a polymer that is insoluble in aqueous media at acidic pH, but soluble at pH above about 5.5. Such a polymer is thus insoluble in gastric fluid, which is acidic, but soluble in intestinal fluid having pH of above 6.0.

Numerous water-insoluble polymers are known in the art including, for example ethylcellulose, cellulose acetate, polyvinyl acetate, and methacrylate copolymers.

Numerous enteric polymers are also known in the art, including, for example, cellulose acetate phthalate, polyvinyl acetate phthalate, hydroxypropyl methylcellulose phthalate, and methyacrylic acid copolymers.

For both water-insoluble polymers and enteric polymers, there are polymer systems available in the form of aqueous latex dispersions that enable film coating without use of organic solvent.

For water-insoluble polymers, known aqueous latex dispersions include, for example, the product sold under the tradename Eudragit NE30D™, which is an aqueous latex dispersion conforming to the monograph for Polyacrylate Dispersion 30% EP (European Pharmacopoeia).

For enteric polymers, known aqueous latex dispersions include, for example, the product sold under the tradename Eudragit L30D-55™, which is an aqueous latex dispersion in conforming to the monograph for Methacrylic Acid Copolymer Dispersion NF.

™ Trademark.

A film-coating that comprises a water-insoluble polymer and an enteric polymer can be applied to core tablets without use of organic solvent by spraying onto the cores a mixture of a latex dispersion of a water-insoluble dispersion and a latex dispersion of an enteric polymer.

In preferred embodiments of the present invention, the film coating will comprise a water-insoluble polymer and an enteric polymer with little if any water-soluble polymer. Preferably, the coating will comprise no water-soluble polymer at all.

The absence of polymer that is soluble in water at acidic pH means that, after ingestion, there will be little, if any, dissolution of tramadol in the acidic gastric fluid. However, when a tablet reaches the small intestine where pH exceeds 6, the enteric polymer in the coating will begin to dissolve, with result that the film coating will become permeable, and the tramadol or salt thereof in the core will begin to permeate through the coating into the intestinal fluid. The rate of release of the tramadol content in intestinal fluid can be controlled by selecting an appropriate ratio of enteric polymer to water-insoluble polymer and a suitable coating thickness.

Tablets of the present invention will preferably meet a dissolution specification as follows, when measured in United States Pharmacopoeia Apparatus #1, at 75 rpm in 900 mL of phosphate buffer at pH 6.8:

Average amount dissolved at 8 hours will be from 20% to 80%, preferably from 30% to 70%, and more preferably from 40% to 60%.

The invention will be better understood from the following examples:

### EXAMPLE 1

Ingredients were mixed in the properties as follows:

| | |
|---|---|
| Tramadol Hydrochloride | 300. |
| Methylcellulose | 43.2 |
| Magnesium Stearate | 1.8 |
| | 345. |

The mixture was compressed into tablets of weight 345 mg per tablet, so that each tablet contained 300 mg of tramadol hydrochloride.

### EXAMPLE 2

The core (i.e. uncoated) tablets from example 1 were then coated in a side-vented coating pan with coating dispersion as follows per kilo of cores:

| | |
|---|---|
| Water | 200. g |
| Talc | 80. g |
| Eudragit NE30D ™ | 160. g |
| Eudragit L30D-55 ™ | 40. g |
| | 480. g |

The coated tablets were placed in an oven overnight at 50° C. for curing of the film coating.

Dissolution of these coated tablets was then tested in USP apparatus #1 at 75 rpm in 900 mL of phosphate buffer pH 6.8. The average dissolution at 8 hours was found to be about 50%, which is very similar to that found for Ultram ER™ tablets 300 mg. These tablets are thus suitable for once-daily administration.

1. A tablet for once-daily oral administration comprising (i) a core comprising tramadol or a salt thereof; and (ii) a coating applied to said core comprising at least one water-insoluble polymer and at least one enteric polymer.

2. A tablet of claim 1 comprising tramadol hydrochloride.

3. A tablet of claim 1 wherein the amount of excipients in the core by weight is less than 50% of the amount of tramadol or salt thereof.

4. A tablet of claim 2 wherein the amount of excipients in the core by weight is less than 30% of the amount of tramadol or salt thereof.

5. A tablet of claim 4 wherein the amount of excipients in the core by weight is less than 20% of the amount of the tramadol or salt thereof.

6. A tablet of claim 1 wherein the water-insoluble polymer comprises a methacrylate copolymer.

7. A tablet of claim 1 wherein the enteric polymer comprises a methacrylic acid copolymer.

8. A tablet of claim 1 made by a process in which the coating is applied as an aqueous latex dispersion.

9. A tablet of claim 1 for which the average amount dissolved at 8 hours is between 20% to 80% when tested in United States Pharmacopoeia Apparatus #1 at 75 rpm in 900 mL of phosphate buffer of pH 6.8.

10. A tablet of claim 1 for which the average amount dissolved at 8 hours is between 30% and 70% when tested in United States Pharmacopoeia Apparatus #1 at 75 rpm in 900 mL of phosphate buffer of pH 6.8.

11. A tablet of claim 1 for which the average amount dissolved at 8 hours is between 40% and 60% when tested in United States Pharmacopoeia Apparatus #1 at 75 rpm in 900 mL of phosphate buffer of pH 6.8.
